(19) United States
(12) Patent Application Publication
(10) Pub. No.: US 2022/0176044 A1
(43) Pub. Date: Jun. 9, 2022

(54) SYRINGE

(71) Applicant: GUANGDONG HAIOU MEDICAL APPARATUS CO., LTD., Puning (CN)

(72) Inventors: Zhuoxuan ZHANG, Puning (CN); Jiangsheng ZHANG, Puning (CN)

(21) Appl. No.: 17/195,667

(22) Filed: Mar. 9, 2021

(30) Foreign Application Priority Data
Dec. 4, 2020 (CN) 202011411516.7

(51) Int. Cl. A61M 5/315; A61M 5/34

(52) U.S. Cl. CPC A61M 5/31513; A61M 5/348

(57) ABSTRACT

A syringe includes a syringe barrel defining a needle receiving chamber and a plunger receiving chamber, a pull-back member received in the needle receiving chamber, and a plunger received in the plunger receiving chamber and comprising an end defining a first receiving chamber and a second receiving chamber. The pull-back member is configured to mount a needle thereto and comprising an engagement portion. The first receiving chamber has a sectional area that is less than a sectional area of the second receiving chamber, and the first receiving chamber has a closed curved surface. The engagement portion is configured to pass through the first receiving chamber and snap-fit into the second receiving chamber when the plunger is inserted into the pull-back member.

### CROSS REFERENCE TO RELATED APPLICATIONS

This application claims priority to Chinese Patent Application No. 202011411516.7, filed Dec. 4, 2020, which is hereby incorporated by reference herein as if set forth in its entirety.

### BACKGROUND

### 1. Technical Field

The present disclosure generally relates to medical devices, and particularly to a syringe.

### 2. Description of Related Art

Syringes are commonly and widely used medical devices, and can be used for injecting drugs into or drawing blood from a human body. In recent years, the public has become increasingly aware of the health hazards associated with needle reuse and accidental needle pickings.

In order to solve the foregoing problems, a great amount of time, effort and money has been invested by syringe manufacturers in developing syringes with safety needle designs. For example, a self-destroying syringe includes biasing structure that is configured to push the needle to deflect, which can prevent reuse of a used syringe. Although the self-destroying syringe can meet basic requirements, it is still desirable and useful to provide a new self-destroying syringe.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is a schematic isometric view of a syringe according to one embodiment.

FIG. 2 is similar to FIG. 1, with the needle deflected from its original position.

FIG. 3 is a planar cross-sectional view of an engagement portion of the syringe of FIG. 1.

FIG. 4 is a planar cross-sectional view of a needle holder of the syringe of FIG. 1.

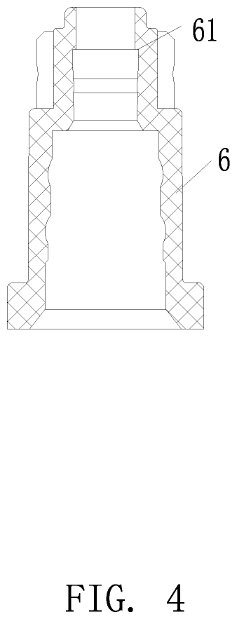

FIG. 5 shows the engagement portion and the needle holder that are assembled together.

FIG. 6 is a planar view of a plunger of the syringe of FIG. 1.

FIG. 7 is similar to FIG. 6, showing a cross section of one end of the plunger.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

The terms “upper”, “lower”, “left” and “right”, indicating the orientational or positional relationship based on the orientational or positional relationship shown in the drawings, are merely for convenience of description, but are not intended to indicate or imply that the device or elements must have a particular orientation or be constructed and operated in a particular orientation, and therefore should not be construed as limiting the present invention. The terms “first” and “second” are used for descriptive purposes only and are not to be construed as indicating or implying relative importance or implicitly indicating the number of technical features. The meaning of “multiple” is two or more, unless expressly stated otherwise.

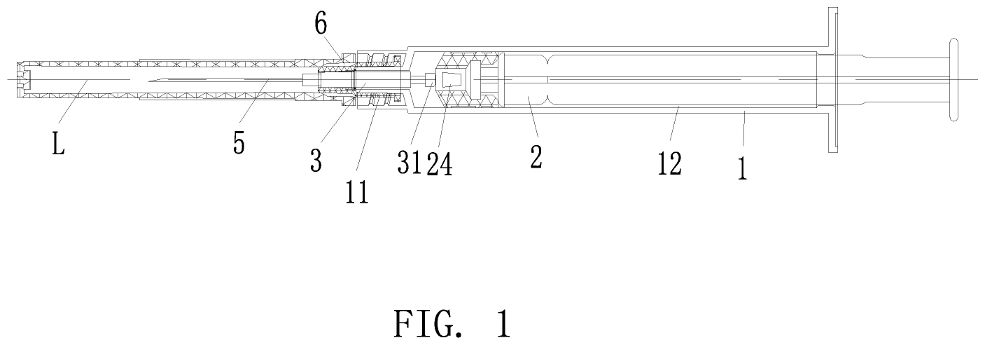

Referring to FIGS. 1-3, in one embodiment, a syringe includes a syringe barrel 1 defining a needle receiving chamber 11 and a plunger receiving chamber 12, a pull-back member 3 received in the needle receiving chamber 11, and a plunger 2 received in the plunger receiving chamber 12 and having an end that defines a first receiving chamber 21 and a second receiving chamber 22. The pull-back member 3 is configured to mount a needle 5 thereto and one end of the pull-back member 3 includes an engagement portion 31. In one embodiment, the pull-back member 3 may define a third receiving chamber 34 and the needle 5 may be received in the third receiving chamber 34. The first receiving chamber 21 has a sectional area that is less than a sectional area of the second receiving chamber 22, and the first receiving chamber 21 includes a closed curved surface. The engagement portion 31 is configured to pass through the first receiving chamber 21 and snap-fit into the second receiving chamber 22 when the plunger 2 is inserted into the pull-back member 3. In one embodiment, an abutment structure 23 may be provided in the engagement portion 31 and/or the second receiving chamber 22. The abutment structure 23 is configured to apply a force to the pull-back member 3 so as to drive the pull-back member 3 and the needle 5 to deflect from an axis L of the syringe barrel 1 when the plunger 2 drives the pull-back member 3 to move.

Since one end of the plunger 2 defines the first receiving chamber 21 and the second receiving chamber 22, and the first receiving chamber 21 has a sectional area that is less than a sectional area of the second receiving chamber 22, a step structure is formed in the plunger 2. When the liquid in the syringe barrel 1 is injected into the body to be injected, the plunger 2 will be pushed toward the pull-back member 3. After the plunger 2 has traveled a predetermined distance, the engagement portion 31 starts to move into the first receiving chamber 21. After passing through the first receiving chamber 21, the engagement portion 31 snap-fits into the second receiving chamber 22, and abuts against the step surface formed between the first receiving chamber 21 and the second receiving chamber 22, which firmly couple the engagement portion 31 to the plunger 2. Since the first receiving chamber 21 is enclosed by a closed curved surface, when the engagement portion 31 of the pull-back member 3 passes through the first receiving chamber 21 and enters the second receiving chamber 22, no elastic deformation occurs at the closed curved surface, so that the engagement portion 31 does not deflect at the first receiving chamber 21. Thus, it is fully guaranteed that when the plunger 2 drives the pull-back member 3 to retreat, the force exerted by the abutment structure 23 completely acts on the pull-back member 3 and the needle 5, so that the pull-back member 3 and the needle 5 can be completely deflect from the axis L of the syringe barrel 1. That is, the needle 5 deflects toward the inner wall of the syringe barrel 1, which effectively achieves the effect of self-destruction, and prevents reuse of the disposable syringe.

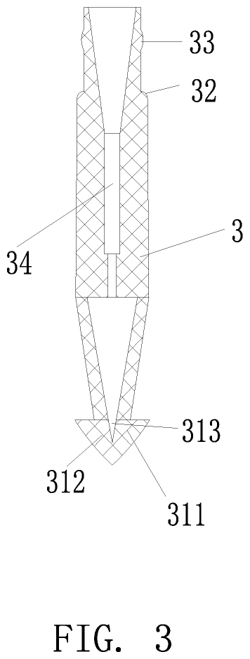

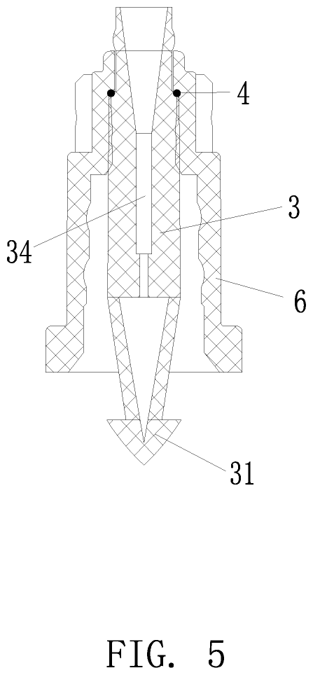

Referring to FIGS. 3 and 5, in one embodiment, the engagement portion 31 comprises a first elastic end 311 and a second elastic end 312, and a gap 313 is formed between the first elastic end 311 and the second elastic end 312. During insertion of the first elastic end 311 and the second elastic end 312 into the first receiving chamber 21, they are pressed to be elastically deformed. The first elastic end 311 and the second elastic end 312 deflect toward each other, which allows the first elastic end 311 and the second elastic end 312 to pass through the first receiving chamber 21 and enter the second chamber 22, and can avoid that a person to be injected feels pricking due to the push generated when the plunger 2 is connected to the pull-back member 3.

In one embodiment, the first elastic end 311 and the second elastic end 312 each may include an inclined surface that enables the engagement portion 31 to pass through the first receiving chamber 21. This arrangement also avoids jamming during the insertion process of the plunger 2. Since the syringe is a part that acts on the human body, the first elastic end 311 and the second elastic end 312 provided with inclined surfaces can prevent persons to be injected from feeling pricking due to the vibration generated when the plunger 2 is connected to the pull-back member 3.

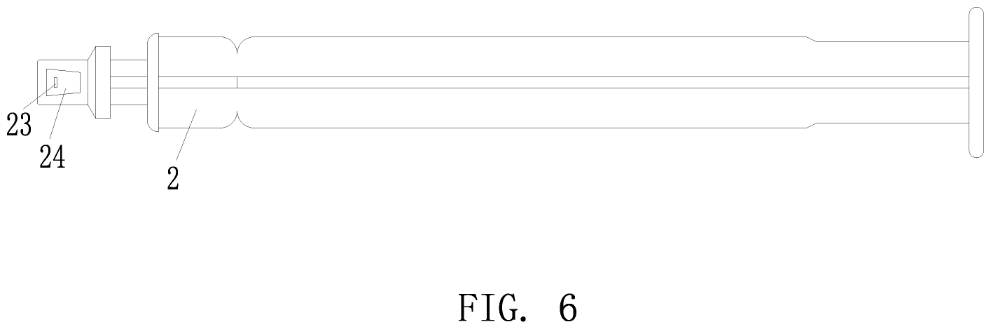

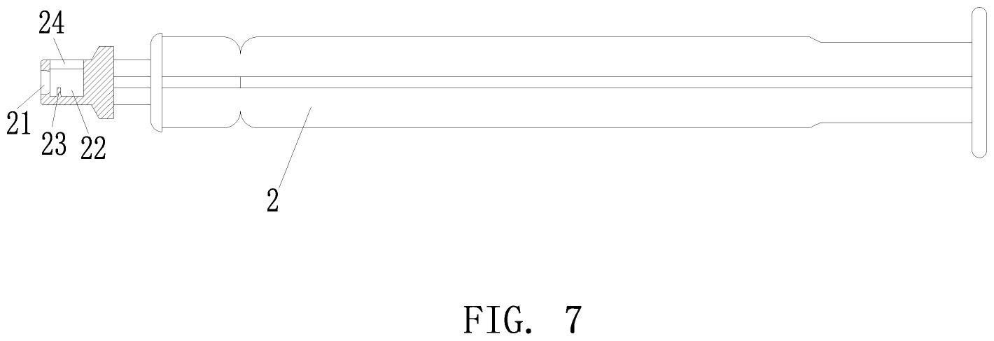

Referring to FIGS. 6 and 7, in one embodiment, an opening 24 may be defined in an inner surface of the second receiving chamber 22, and the first elastic end 311 and the second elastic end 312 are snap-fitted in the second receiving chamber 22. In this way, the first elastic end 311 and the second elastic end 312 are further fixed, that is, the pull-back member 3 is fixed to the plunger 2, thereby ensuring the stability of the connection and strength of the connection.

The opening 24 provides a receiving space for the first elastic end 311 and the second elastic end 312. In one embodiment, the opening 24 can be a blind hole. That is, the opening 24 may not pass through the lateral wall of the second receiving chamber 22.

In another embodiment, the opening 24 can be a through hole. That is, the opening 24 may not pass through the lateral wall of the second receiving chamber 22. This configuration can better fix the first elastic end 311 and the second elastic end 312, thereby ensuring the stability of the connection.

In one embodiment, the abutment structure 23 is a protrusion formed on the inner surface of the second receiving chamber 22. When the engagement portion 31 is inserted into the second receiving chamber 22, the engagement portion 31 is pressed by the protrusion (i.e., the abutment structure 23), so that the protrusion exerts a force on the engagement portion 31 when the plunger 2 drives the pull-back member 3 to retreat, which causes the pull-back member 3 and the needle 5 to deflect from the axis L of the needle 5, thereby achieving the self-destruction of the syringe.

In another embodiment, the abutment structure 23 may be a protruding boss provided on an inclined surface of one of the first elastic end 311 and the second elastic end 312.

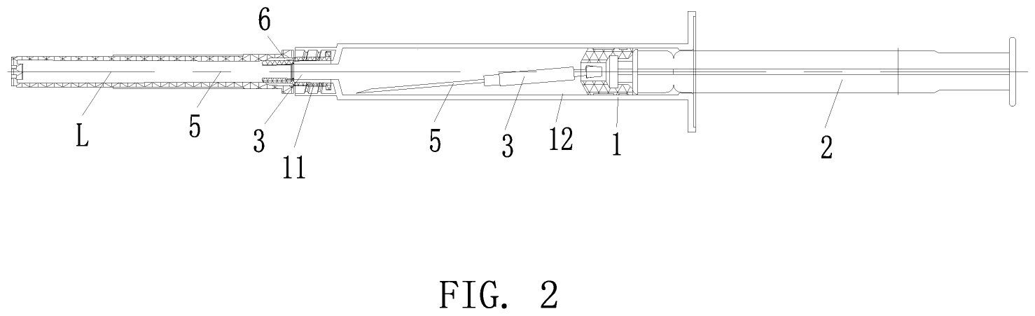

Referring again to FIGS. 1 and 2, in one embodiment, the syringe may further include a needle holder 6 arranged on an outside of one end of the syringe barrel 1 and tightly connected with the pull-back member 3 in a tight-fitting manner. With such configuration, the needle holder 6 cooperates with the syringe 1 to hold the pull-back member 3 in position, which avoid that normal injection is adversely affected due to the deflection of the pull-back member 3 when the end of the plunger 2 enters the pull-back member 3. The needle holder 6 plays a role in restricting the pull-back member 3 and positions the pull-back member 3.

In one embodiment, the pull-back member 3 includes a first protrusion 32, and the needle holder 6 includes a second protrusion 61 on an inner surface thereof. The second protrusion 61 is configured to prevent the pull-back member 3 from moving toward an inside of the syringe barrel 1 by a force from the needle 5, which prevents the pull-back member 3 connected to the needle 5 from being pushed out due to the excessive pushing force applied to the plunger 2.

In one embodiment, a sealing structure 4 is arranged around the first protrusion 32. The sealing structure 4 may be a sealing O-ring. The arrangement of the sealing ring prevents the injection liquid from leaking from the connection between the needle holder 6 and the pull-back member 3, thereby protecting the sealing performance of the syringe.

In one embodiment, the pull-back member 3 may include a protruding portion 33 on an outer surface of one end away from the engagement portion 31. The protruding portion 33 is provided to counteract the reaction force when the syringe is injecting a person.

Referring again to FIG. 1, when the syringe is in use, before the injection is completed, the axis of the pull-back member 3 coincides with the axis L of the syringe barrel 1 by the supporting force of the syringe barrel 1 and the needle holder 6. During injection, the plunger 2 is pushed by an external force and moves toward the human body. The engagement portion 31 of the pull-back member 3 passes through the first receiving chamber 21 of the plunger 2, and enters the second receiving chamber 22. During the entering process of the engagement portion 31, the first elastic end 311 and the second elastic end 312 are compressed and contracted by the closed curved surface at the end of the plunger 2. Since there is a gap 313 between the first elastic end 311 and the second elastic end 312, it is convenient for the engagement portion 31 to smoothly enter the second receiving chamber 22. After the engagement portion 31 is completely inserted into the second receiving chamber 22, the first elastic end 311 and the second elastic end 312 receive no compression force and starts to return to their initial state. After entering the second receiving chamber 22, the engagement portion 31 can no longer withdraw from the second receiving chamber 22, thereby achieving the interlocking of the end of the plunger 2 and the pull-back member 3. Referring again to FIG. 2, after the injection is completed, the plunger 2 can be pulled back. Since one end of the plunger 2 has been locked with the pull-back member 3, the pull-back member 3 will move back with the plunger 2. After the pull-back member 3 loses the support from the needle holder 6 and the syringe barrel 1, the abutment structure 23 in the push rod 2 applies a pushing force to the engagement portion 31 of the pull-back member 3, and the pull-back member 3 and the needle 5 deflect together. The axis of the pull-back member 3 and the needle 5 do not coincide with the axis L of the syringe barrel 1, so that the needle 5 will not be pushed out of the syringe barrel 1 again, which can avoid the risk of disease transmission due to the reuse of the syringe.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A syringe comprising:
a syringe barrel defining a needle receiving chamber (11) and a plunger receiving chamber (12);
a pull-back member (3) received in the needle receiving chamber (11), the pull-back member (3) configured to mount a needle (5) thereto and comprising an engagement portion (31); and
a plunger (2) received in the plunger receiving chamber (12) and comprising an end defining a first receiving chamber (21) and a second receiving chamber (22), the first receiving chamber (21) having a sectional area that is less than a sectional area of the second receiving chamber (22), and the first receiving chamber (21) comprising a closed curved surface;
wherein the engagement portion (31) is configured to pass through the first receiving chamber (21) and snap-fit into the second receiving chamber (22) when the plunger (2) is inserted into the pull-back member (3).

2. The syringe according to claim 1, wherein the engagement portion (31) comprises a first elastic end (311) and a second elastic end (312), and a gap (313) is defined between the first elastic end (311) and the second elastic end (312).

3. The syringe according to claim 2, wherein the first elastic end (311) and the second elastic end (312) each comprise an inclined surface that enables the engagement portion (31) to pass through the first receiving chamber (21).

4. The syringe according to claim 2, wherein an opening (24) is defined in an inner surface of the second receiving chamber (22), and the first elastic end (311) and the second elastic end (312) are snap-fitted in the second receiving chamber (22).

5. The syringe according to claim 4, further comprising an abutment structure (23), wherein the abutment structure (23) is provided in the engagement portion (31) and/or the second receiving chamber (22), the abutment structure (23) is configured to apply a force to the pull-back member (3) so as to drive the pull-back member (3) and the needle (5) to deviate from an axis of the syringe barrel (1) when the plunger (2) drives the pull-back member (3) to move.

6. The syringe according to claim 5, wherein the abutment structure (23) is a protrusion formed on the inner surface of the second receiving chamber (22), or a protruding boss provided on an inclined surface of one of the first elastic end (311) and the second elastic end (312).

7. The syringe according to claim 1, further comprising a needle holder (6) arranged on an outside of one end of the syringe barrel (1) and tightly connected with the pull-back member (3) in a tight-fitting manner.

8. The syringe according to claim 7, wherein the pull-back member (3) comprises a first protrusion (32), the needle holder (6) comprises a second protrusion (61) on an inner surface thereof, the second protrusion (61) is configured to prevent the pull-back member (3) from moving toward an inside of the syringe barrel (1) by a force from the needle (5).

9. The syringe according to claim 8, further comprising a sealing structure (4) arranged around the first protrusion (32).

10. The syringe according to claim 9, wherein the pull-back member (3) comprises a protruding portion (33) on an outer surface of one end away from the engagement portion (31).
